(19) United States
(12) Patent Application Publication
(10) Pub. No.: US 2005/0103349 A1
(43) Pub. Date: May 19, 2005

(54) Male condom

(76) Inventor: Chantal Couvreur, Kraainem (BE)

Correspondence Address: CANTOR COLBURN, LLP, 55 GRIFFIN ROAD SOUTH, BLOOMFIELD, CT, 06002, US

(21) Appl. No.: 10/505,058

(22) PCT Filed: Jan. 13, 2003
(86) PCT No.: PCT/EP03/00223

(30) Foreign Application Priority Data
Feb. 20, 2002 (LU) 90 891

(51) Int. Cl. A61F 6/04

(52) U.S. Cl. 128/844; 128/842

(57) ABSTRACT

Condom for protection against the transfer of sexually transmitted diseases, comprising a flexible thin-walled tube having a closed end and an open end, said open end having a means for stretching it radially, which comprises a collarette extending in a direction perpendicular to said tube so as to cover the base of the penis.

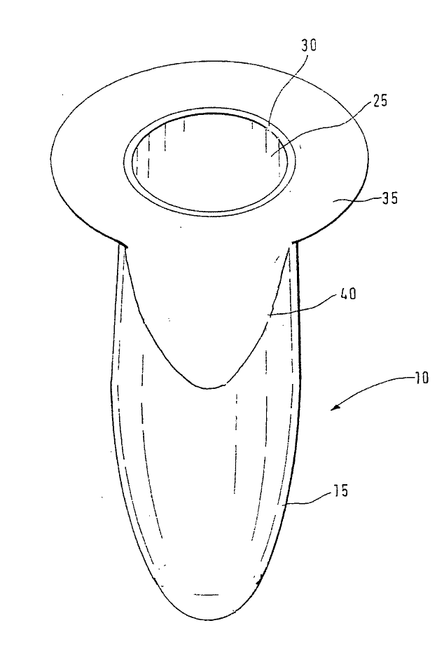

The present invention relates to male condoms.

While the incidence of AIDS among homosexuals and bisexual men has been observed to stabilize, the proportion of the heterosexual population infected is still increasing—most of these cases arise from indulgence in sexual relations without taking precautions.

The fight against AIDS and other sexually transmitted diseases (STDs) is a major challenge. One fifth of the adult American population for example suffers from a sexually transmitted disease, this being a cause of infertility or cancer, and increases the vulnerability to AIDS.

The condom is the best barrier against these diseases, but its effectiveness depends, among other things, on that part of the body that is covered and on the way it is used.

To be effective, it is necessary for the man to withdraw immediately after ejaculation so that the condom does not fall off when the penis softens. The man should always withdraw while his penis is still erect so that the sperm cannot spill out into the vagina or anus. As this reduces pleasure, this practice is not automatically respected.

At the present time, there are a number of condoms on the market.

One of the disadvantages of the male condom is that the man must withdraw immediately after ejaculation in order to prevent sperm from flowing between the penis and the condom and coming into contact with his partner's mucous membranes.

It is an object of the present invention to propose a condom that reduces the risk of transmitting sexually transmitted diseases. It is also an object of the present invention to reduce the risk of passing sexual fluids between partners.

For this purpose, the invention proposes a condom characterised in that the said means for stretching radially the open end comprises a collarette or rim extending in a direction perpendicular to said tube so as to cover the base of the penis during intercourse.

Conventional condoms, even if they are very long, do not in general cover the erect penis over its entire length and there is a risk of the base of the penis, which is not covered by the condom, coming into contact with the woman's body fluids in case of penetration beyond the limit of the condom. At that moment, it is not possible for the base of the condom to be held manually during withdrawal after ejaculation.

The collarette covering the base of the penis keeps the condom in place and is an effective way of preventing the transmission of diseases since contact between the partners' body fluids is prevented. The collarette forms an effective barrier and prevents the penis from penetrating too deeply.

Since the collarette remains outside of the vulva or of the anus, the man is able to hold the condom after ejaculation during withdrawal of his penis. Even if sperm were to flow out between the penis and the condom, it would not come into contact with his partner's mucous membranes owing to the presence of the collarette. This constitutes a considerable advantage over known condoms that do not have this collarette, with which it may happen that the condom remains jammed inside the vagina or inside the anus after the penis has been slid out after ejaculation.

According to a first advantageous embodiment, a means to radially stretch open end comprises a ring that has roughly the same diameter as said tube and that delimits the collarette with respect to the tube. The open end of the tube is kept open using, for example, a plastic ring which includes, around its outer perimeter, a collarette.

Advantageously, the collarette includes an elongate protuberance or tongue extending toward the scrotum. Preferably, this tongue covers the region lying between the vulva and the anus of the woman during intercourse.

Advantageously, the collarette is made from a more rigid material and/or a thicker material than the thin flexible tube.

In a preferred embodiment, the length of the flexible thin-walled tube is chosen so that the collarette is maintained in contact with the base of the penis. With a view to increasing the comfort of the condom, it is preferable for the collarette to be maintained permanently pressed against the scrotum and the pubis. As penises may have different sizes, it is preferable to make condoms available in various lengths. It might also be possible to provide various widths of collarette.

In a preferred embodiment, the collarette is maintained and the tube are made in one piece. Alternatively, the collarette and the tube are made in two separate pieces. The collarette may then comprise a means for fixing said thin-walled tube like for example a groove in which the open end of the tube is lodged.

Advantageously, the flexible thin-walled tube is made from a polymeric material such as natural rubber latex (NRL), polyethylene, polyurethane and derivatives and blends of these materials. The condom may be lubricated inside and/or outside with a lubricant suitable for improving comfort.

Other features of the invention are described, in a non-limiting way, in the examples and in relation to the FIGURE which shows a condom.

The condom 10 into which the erect penis can be inserted comprises a flexible thin-walled tube 15 having a closed end 20 and an open end 25. The open end 25 comprises a ring 30 that has roughly the same diameter as said tube 15 and that delimits the collarette 35 with respect to the tube 15. The open end 25 of the tube 15 is kept open using this ring 30, which includes, on its outer perimeter, the collarette 35 made of a harder material to the touch than the thin flexible tube. The collarette 35 extends in a direction perpendicular to said tube 15 so as to cover the base of the penis. The condom 15 also offers good protection against the transmission of sexually transmitted diseases as well as efficient protection against unintentional pregnancies. The collarette, which extends substantially perpendicular to the base of the penis and reduces the risk of an overly deep penetration beyond that part of the penis that is covered by the thin tube, makes the condom safer than a standard male condom. This ring keeps the condom in position during and after intercourse. The condom is kept in place on the penis during intercourse, and likewise the collarette does not in principle move.

The thickness of the tube and of the collarette may vary, it being understood that greater wall and collarette thicknesses reduce sensitivity during intercourse. Typical thicknesses for the tube lie between 20 and 100 microns, while the collarette may be thicker.

Of course, the wall thickness must be such that the resistance to mechanical stresses of the condom laid down by standards in force in the different countries is guaranteed. The dimensions of the condom according to the invention are in principle identical to the dimensions of standard condoms.

The collarette 35 includes an elongate protuberance or tongue 40 extending toward the anus, which is placed parallel to the vulva and perpendicular to the tubular part of the condom. The tongue 40 covers the user's scrotum and the region lying between the woman's vulva and anus during intercourse.

The condom 10 may be produced in various lengths, i.e. the length of the tube 15 may be different depending on the size of the user's penis, so that for each user the collarette 35 is maintained in contact with the base of the penis and the pubis. In general, the width of the collarette 35 lies between 1 and 4 cm and preferably between 1 and 2 cm. Of course, the width of the collarette 35 need not necessarily be uniform over especially its perimeter.

The condom 15 is made from a polymeric material such as natural rubber latex (NRL), polyethylene, polyurethane and derivatives and blends of these materials. The condom 15 may be lubricated inside and/or outside with a lubricant sufficient to improve the comfort.

Unlike a standard male condom, it is unnecessary to remove the condom according to the invention immediately after ejaculation.

1. Male condom for protection against the transfer of sexually transmitted diseases and able to be placed on the penis of a user, the condom comprising:
a flexible thin-walled tube having a closed end and an open end, said open end having a means for stretching said open end radially; and
a collarette extending in a direction perpendicular to said tube so as to cover the base of the penis during intercourse;
wherein said collarette and said tube are made from two separate parts.

2. Condom as claimed in claim 1, wherein the means for stretching said open end radially comprises a ring that has a diameter substantially the same as a diameter of said tube, the ring delimiting the collarette with respect to the tube.

3. Condom as claimed in claim 1, wherein the collarette includes an elongate protuberance extending toward the scrotum of the user.

4. Condom as claimed in claim 1, wherein the collarette is made from a more rigid material than the thin flexible tube.

5. Condom as claimed in claim 1, wherein the collarette is made from a thicker material than the thin flexible tube.

6. Condom as claimed in claim 1, wherein the flexible thin-walled tube includes a length such that the collarette is maintained in contact with a base of the penis.

7-8. (canceled)

9. Condom as claimed in claim 1, wherein said collarette includes a means for fixing said thin-walled tube.

10. Condom as claimed in claim 9, wherein said means for fixing said thin-walled tube comprises a groove.

11. Condom as claimed in claim 1, wherein the flexible thin-walled tube is made from a polymeric material.

12. Condom as claimed in claim 1, wherein the flexible thin-walled tube is made from at least one of a natural rubber latex (NRL), a polyethylene, a polyurethane, or a derivative or blend thereof.
